(19) United States
(12) Patent Application Publication
(10) Pub. No.: US 2008/0031980 A1
(43) Pub. Date: Feb. 7, 2008

(54) Curcumin-containing composition, methods of making, and methods of using

(76) Inventors: Al Rodriguez, Northridge, CA (US); Daniel Shoskes, Moreland Hills, OH (US)

Correspondence Address: Law Office of John K. Pike, PLLC, 2121 Eisenhower Avenue, Suite 200, Alexandria, VA, 22314, US

(21) Appl. No.: 11/497,319

(22) Filed: Aug. 2, 2006

(51) Int. Cl. A61K 36/00; A61P 13/00; A61P 15/00

(52) U.S. Cl. 424/773

(57) ABSTRACT

A composition includes 50-80 wt. % of at least one curcumin compound, 10-50 wt. % quercetin, ≧3 wt. % papain, and ≧3 wt. % bromelain, wherein the composition is suitable for oral administration to a human. Methods of making and using the composition are provided.

### DETAILED DESCRIPTION OF THE SEVERAL EMBODIMENTS

One embodiment of the present invention provides a composition for treating one or more maladies such as chronic nonbacterial prostatitis, chronic pelvic pain syndrome, overactive bladder, sexual dysfunction, prostatitis, prostatodynia, urinary incontinence, pain during intercourse, genitourinary pain not associated with infection, bladder pain, abdominable pain, urinary frequency, category III chronic prostatitis syndrome, category IIIa chronic pelvic pain syndrome, category IIIb chronic pelvic pain syndrome, chronic voiding symptoms not associated with infection, adverse side effects associated with renal transplantation, protection against kidney damage associated with the administration of aminoglycosides, delayed graft function in kidney transplant patient, acute rejection in kidney transplant patient, drug induced neuropathy in kidney transplant patient, drug induced tremor in kidney transplant patient, chronic allograft nephropathy in kidney transplant patient, post-transplant risk of colon cancer in kidney transplant patient, post-transplant risk of prostate cancer in kidney transplant patient, protection against kidney damage from intravenous contrast dye, protection against ischemic kidney damage from trauma or surgery, hypercholesterolaemia, high LDL cholesterol level, or a combination thereof.

The composition desirably includes:

50-80 wt. % of at least one curcumin compound;

10-50 wt. % quercetin;

≧3 wt. % papain; and

≧3 wt. % bromelain;

wherein the composition is suitable for oral administration to a human.

The composition is effective in treating the maladies listed herein.

The term, “wt. %” is intended to mean weight percent of the subject ingredient based on the weight of the composition.

The composition includes 50-80 wt. % of at least one curcumin compound. This range includes all values and subranges therebetween, including 50, 51, 52, 53, 54, 55, 56, 57, 58, 59 60, 61, 62, 63, 64, 65, 66, 67, 68, 69, 70, 71, 72, 73, 74, 75, 76, 77, 78, 79, and 80 wt. %. Some examples of non-limiting subranges include about 60-70 wt. % of the curcumin compound.

In one embodiment, the composition includes about 65 wt. % of the curcumin compound.

The curcumin compound may be one or more of bisdemethoxycurcumin, demethoxycurcumin, curcumin, dihydrocurcumin, tetrahydrocurcumin, hexahydrocurcumin, octahydrocurcumin, diacetylcurcumin, p, p dihydroxydicinnamoylmethane, hexahydrocurcuminol, curcumin glucuronide, curcumin sulfate, [6]-paradol, dihydrocapsaicin, salicylcurcuminoid, monomethylcurcumin, trimethylcurcumin, 1,7-bis(3,4-dihydroxyphenyl)-11,6-heptadiene-3,5-dione, p-dyhydroxydicinamoyl feruloyl methane, capsaicin, [6]-gingerol, cassumunin A, cassumunin B, yakuchinone A, yakuchinone B, isoeugenol, dibenzoylmethane, rosmarinic acid, 2-hydroxydibenzoylmethane, hydrazinocurcumin, hydrazinodemethoxycurcumin, hydrazinobisdemethoxycurcumin, hydrazinobenzoyldemethoxycurcmin, and a combination thereof.

In one embodiment, the curcumin compound is one or more of bisdemethoxycurcumin, demethoxycurcumin, curcumin, or a combination thereof. In another embodiment, the curcumin compound is a combination of bisdemethoxycurcumin, demethoxycurcumin, and curcumin.

In one embodiment, the curcumin compound is CURCUMIN C3 COMPLEX®, available from Sabinsa Corp., a combination of of bisdemethoxycurcumin, demethoxycurcumin and curcumin. Some curcumin compounds are disclosed, for example, in U.S. Pat. No. 5,861,415, incorporated herein by reference.

The composition includes 10-50 wt. % quercetin. This range includes all values and subranges therebetween, including 10, 11, 12, 13, 14, 15, 16, 17, 18, 19, 20, 21, 23, 25, 27, 29, 30, 31, 33, 35,37, 39, 40, 41, 43, 45, 47, 49, and 50 wt. %. Some examples of non-limiting subranges include about 15-45, about 25-40, and about 25-30 wt. % quercetin.

In one embodiment, the composition includes about 25-30 wt. % quercetin.

The composition includes ≧3 wt. % papain. This range includes all values and subranges therebetween, including ≧3, 3.0, 3.1, 3.2, 3.3, 3.4, 3.5, 3.6, 3.7, 3.8, 3.9, 4, 5, 6, 7, 8, 9, 10, 11, 12, 13, 14, 15, 16, 17, 18, 19, 20 and higher wt. % papain. Some examples of non-limiting subranges include about 3.1-20 and about 3.1-5.1 wt. % papain.

In one embodiment, the composition includes about 3.1-5.1 wt. % papain.

The composition includes ≧3 wt. % bromelain. This range includes all values and subranges therebetween, including ≧3, 3.0, 3.1, 3.2, 3.3, 3.4, 3.5, 3.6, 3.7, 3.8, 3.9, 4, 5, 6, 7, 8, 9, 10, 11, 12, 13, 14, 15, 16, 17, 18, 19, 20, and higher wt. % bromelain. Some non-limiting examples of subranges include about 3.1-10 and 3.1-5.1 wt. % bromelain.

In one embodiment, the composition includes about 3.1-5.1 wt. % bromelain.

The composition may optionally additionally include one or more of cranberry, green tea extract, black cohosh, wood betony, passion flower, valerian root, saw palmetto, zinc, calcium, or a combination thereof.

The composition may optionally include cranberry in an amount of 0.5-5 wt. %. This range includes all values and subranges therebetween, including 0.5, 0.6, 0.7, 0.8, 0.9, 1, 2, 3, 4, and 5 wt. %.

The composition may optionally include green tea extract in an amount of 1-15 wt. %. This range includes all values and subranges therebetween, including 1, 2, 3, 4, 5, 6, 7, 8, 9, 10, 11, 12, 13, 14, and 15 wt. % green tea extract.

The composition may optionally include black cohosh in an amount of 0.1-5 wt. %. This range includes all values and subranges therebetween, including 0.1, 0.2, 0.3, 0.4, 0.5, 0.6, 0.7, 0.8, 0.9, 1, 2, 3, 4, and 5 wt. %.

The composition may optionally include wood betony in an amount of 0.1-5 wt. %. This range includes all values and subranges therebetween, including 0.1, 0.2, 0.3, 0.4, 0.5, 0.6, 0.7, 0.8, 0.9, 1, 2, 3, 4, and 5 wt. %.

The composition may optionally include passion flower in an amount of 0.1-5 wt. %. This range includes all values and subranges therebetween, including 0.1, 0.2, 0.3, 0.4, 0.5, 0.6, 0.7, 0.8, 0.9, 1, 2, 3, 4, and 5 wt. %.

The composition may optionally include valerian root in an amount of 0.1-5 wt. %. This range includes all values and subranges therebetween, including 0.1, 0.2, 0.3, 0.4, 0.5, 0.6, 0.7, 0.8, 0.9, 1, 2, 3, 4, and 5 wt. %.

The composition may optionally include saw palmetto in an amount of 0.5-5 wt. %. This range includes all values and subranges therebetween, including 0.5, 0.6, 0.7, 0.8, 0.9, 1, 2, 3, 4, and 5 wt. %.

The composition may optionally include zinc in an amount of 0.1-15 wt. %. This range includes all values and subranges therebetween, including 0.1, 0.2, 0.3, 0.4, 0.5, 0.6, 0.7, 0.8, 0.9, 1, 2, 3, 4, 5, 6, 7, 8, 9, 10, 11, 12, 13, 14, and 15 wt. %.

The zinc may be in the form of a salt. Some examples of non-limiting zinc salts include zinc sulfate, zinc acetate, zinc gluconate, zinc lactate, zinc salicylate, zinc stearate, zinc tannate, zinc tartrate, zinc carnosine, zinc L-carnosine, zinc picolinate, and/or zinc citrate. Mixed salts and combinations of zinc salts are possible.

The composition may optionally include calcium in an amount of 1-10 wt. %.This range includes all values and subranges therebetween, including 1, 2, 3, 4, 5, 6, 7, 8, 9, and 10 wt. %.

The calcium may be present in the form of a salt. Some examples of non-limiting calcium salts include dibasic calcium phosphate, tribasic calcium phosphate, calcium fumarate, calcium citrate, calcium maleate, calcium lactate, calcium lactate gluconate, calcium gluconate, calcium acetylsalicylate, calcium ascorbate, calcium carbonate, calcium levulinate, calcium pantothenate, and/or hydrates thereof. Combinations of salts and/or hydrates are possible.

The composition may be suitably administered to treat one or more of chronic nonbacterial prostatitis, chronic pelvic pain syndrome, overactive bladder, sexual dysfunction, prostatitis, prostatodynia, urinary incontinence, pain during intercourse, genitourinary pain not associated with infection, bladder pain, abdominable pain, urinary frequency, category III chronic prostatitis syndrome, category IIIa chronic pelvic pain syndrome, category IIIb chronic pelvic pain syndrome, chronic voiding symptoms not associated with infection, adverse side effects associated with renal transplantation, protection against kidney damage associated with the administration of aminoglycosides, delayed graft function in kidney transplant patient, acute rejection in kidney transplant patient, drug induced neuropathy in kidney transplant patient, drug induced tremor in kidney transplant patient, chronic allograft nephropathy in kidney transplant patient, post-transplant risk of colon cancer in kidney transplant patient, post-transplant risk of prostate cancer in kidney transplant patient, protection against kidney damage from intravenous contrast dye, protection against ischemic kidney damage from trauma or surgery, hypercholesterolaemia, high LDL cholesterol level, and a combination thereof.

The human may be a male or a female. The human may be at risk for one or more of the maladies herein, may be suspected of having one or more of the maladies herein, or may be known to have one or more of the maladies herein. These are known maladies, and one of ordinary skill, such as a diagnosing physician, can determine whether a subject human is at risk for having or contracting, suspected of having, or has one or more of the maladies herein. In addition, the subject human may self-diagnose several of the maladies herein given their symptoms and using the information available to the public, for example, in a library, a self-help group, or over the Internet.

The composition may be formed by contacting:

50-80 wt. % of at least one curcumin compound;

10-50 wt. % quercetin;

≧3 wt. % papain; and

≧3 wt. % bromelain;

to form a composition suitable for oral administration to a human.

The balance to 100 wt. %, if any, may be made up with one or more excipients, additional ingredients, and the like, as appropriate.

The contacting, capsule filling, and/or tabletting may be carried out according to known methods. In one embodiment, the curcumin compound, quercetin, papain, and bromelain, and, optionally one or more excipients, e.g., diluent, binder, glidant, lubricant, and/or disintegrant, are combined in appropriate amounts, then filled into a capsule, such as a gelatin capsule. In another embodiment, they are tabletted and optionally coated with one or more sustained release agent or agent. Some examples of formulating methods may be found in, Remington: The Science and Practice of Pharmacy, 21st Ed., (2005), the entire contents of which are hereby incorporated by reference.

Referring to the maladies described herein, the terms, “treat”, “treating” and “treatment”, as used herein refer to reduction in severity and/or frequency of symptoms, elimination of symptoms and/or underlying cause, prevention of the occurrence of symptoms and/or their underlying cause, and/or improvement or remediation of damage. This may include prevention of a particular disorder or unwanted physiological event as well as treatment of a clinically symptomatic individual by inhibiting or causing regression of a disorder or disease. It is understood that treating may effect even only a slight improvement or delay of onset and not a total cure or total prevention.

By the term “effective amount” is meant a nontoxic but sufficient amount of a beneficial agent or agents to provide the desired treating effect. The amount of beneficial agent or agents that is effective may vary from subject to subject, depending on the age and general condition of the individual, the particular beneficial agent or agents, and the like. Thus, it is not always possible to specify an exact effective amount. However, an appropriate effective amount in any individual case may be determined by one of ordinary skill in the art using routine experimentation and given the teachings herein. In one embodiment, the composition includes effective amounts of one or more of the active agents.

The term “sustained release” is used in its conventional sense to mean that the composition provides for gradual release of a beneficial agent or agents over an extended period of time. See, for example, Remington: The Science and Practice of Pharmacy, 21st Ed., (2005), already incorporated by reference. In one embodiment, the sustained release results in substantially constant blood and/or localized level of the agent or agents over an extended period.

In one embodiment, the extended period over which one or more of the active agent or agents are released from the composition in vivo or in vitro may range from 1 to 18 hours. This range includes all values and subranges therebetween, including 1, 2, 3, 4, 5, 6, 7, 8, 9, 10, 11, 12, 13, 14, 15, 16, 17, and 18 hours.

In one embodiment, the composition includes one or more sustained release agents. The sustained release agent or agents may be in the form of a coating, an admixed compound, or a combination of coating and admixture. In one embodiment, the composition may be in the form of dried granules and/or nonpareils, which have been individually coated with a coating or a sustained release coating, and which are filled into a hard gelatin capsule or compacted into a tablet.

The sustained release may be effected, for example, when the composition is in tablet or capsule form. The tablets or capsules may respond to some physiological condition to release the agents through, for example, one or more coatings, enteric coatings, or admixed agents. In another embodiment, the sustained release may be effected via a combination of mechanisms. In one embodiment, the sustained release may occur in a steady, controlled manner. In another embodiment, the sustained release dissolution profile may be continuous or discontinuous. For example, the release of the active agent or agents may occur in pulses.

The sustained release agent may be in the form of a coating. Some non-limiting examples of sustained release coatings include gelatin, cellulose acetate phthalate, cellulose ether, cellulose acetate butyrate, cellulose acetate, cellulose diacetate, cellulose triacetate, poly(lactic-co-glycolic acid), beeswax, carnauba wax, glyceryl monostearate, stearic acid, palmitic acid, glyceryl monopalmitate, cetyl alcohol, shellac, zein, silicone elastomer, acrylic resin, acrylic acid copolymer, acrylic acid ester copolymer, methacrylic acid copolymer, methacrylic acid ester copolymer, copolymer of esters of acrylic and methacrylic acid, ethylcellulose, ethylcellulose ether, polyvinylacetate stabilized with povidone and sodium laurylsulfate, polyethylacrylate, polymethyl methacrylate, methylcellulose, hydroxypropyl methylcellulose, copolymers thereof, blends thereof, and/or a combination thereof.

Other examples of non-limiting sustained release coatings include EUDRAGIT® NE 30D, NE 40D, RL 30D, and RS 30D (copolymers derived from esters of acrylic and methacrylic acid) available from Roehm; SURELEASE® (ethylcellulose) and METHOCEL® (ethylcellulose ether) both available from Colorcon; KOLLICOAT® SR 30 D (polyvinylacetate dispersion stabilized with povidone and sodium laurylsulfate) and KOLLICOAT® EMM 30 D (poly (ethylacrylate, methyl methacrylate)) both available from BASF; AQUACOAT® CPD (cellulose acetate phthalate) and AQUACOAT® ECD (ethylcellulose) both available from FMC; ETHOCEL® Standard 7, 10, or 20 Premium (ethylcellulose), METHOCEL® water-soluble methylcellulose and hydroxypropyl methylcellulose, METHOCEL® K Premium, METHOCEL® K100LV Premium, METHOCEL® K4M Premium, METHOCEL® K15M Premium, METHOCEL® K100M Premium, METHOCEL® E4M Premium, METHOCEL® E10M Premium, METHOCEL® Premium blended with water-insoluble ETHOCEL products, METHOCEL® E5 Premium, and METHOCEL® E15 Premium, available from Dow Chemical

The sustained release agent may be present in an amount of 0.1 to 10 wt. %. This range includes all values and subranges therebetween, including 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, and 10 wt. %.

In one embodiment, the sustained release coating may be a mixture of wax, for example beeswax and/or carnauba wax, with a combination of glyceryl monostearate, stearic acid, palmitic acid, glyceryl monopalmitate, and cetyl alcohol.

In one embodiment, the sustained release agent is in the form of an ethylcellulose coating on the tabletted composition.

The composition may contain one or more diluents. Some non-limiting examples of diluents include dicalcium phosphate, calcium sulfate, lactose, cellulose, kaolin, mannitol, sodium chloride, dry starch, powdered sugar, sorbitol, sucrose, inositol, microcrystalline cellulose, bentonite, and the like. Combinations are possible. The diluent or diluents may be present in an amount ranging from 0.5 to 20 wt. %. This range includes all values and subranges therebetween, including 0.5, 1, 2, 3, 4, 5, 6, 7, 8, 9, 10, 11, 12, 13, 14, 15, 16, 17, 18, 19, and 20 wt. %.

The composition may contain one or more binders. Some non-limiting examples of binders include starch, gelatin, sugar, sucrose, glucose, dextrose, molasses, lactose, natural gum, synthetic gum, acacia, sodium alginate, extract of Irish moss, panwar gum, ghatti gum, mucilage of isapol husks, carboxymethylcellulose, methylcellulose, polyvinylpyrrolidone, microcrystalline cellulose, microcrystalline dextrose, amylase, starch paste, corn starch, gelatin, hydroxypropyl methylcellulose, hydroxyethylcellulose, veegum, larch arabogalactan, polyethylene glycol, ethylcelluolose, water, alcohol, and the like. Combinations are possible. The binder or binders may be present in an amount ranging from 1 to 20 wt. %. This range includes all values and subranges therebetween, including 1, 2, 3, 4, 5, 6, 7, 8, 9, 10, 11, 12, 13, 14, 15, 16, 17, 18, 19, and 20 wt. %.

The composition may contain one or more lubricants. Some non-limiting examples of lubricants include talc, magnesium stearate, calcium stearate, stearic acid, aluminum stearate, glyceryl behanate, hydrogenated vegetable oil, polyethylene glycol, sodium benzoate, sodium acetate, sodium chloride, leucine, Carbowax, and the like. Combinations are possible. The lubricant or lubricants may be present in an amount ranging from 0.1 to 5 wt. %. This range includes all values and subranges therebetween, including 0.1, 0.2, 0.3, 0.4, 0.5, 0.6, 0.7, 0,8, 0.9, 1, 2, 3, 4, and 5 wt. %.

The composition may contain one or more glidants. Some non-limiting examples of glidants include colloidal silicon dioxide, talc, and the like. Combinations are possible. The glidant or glidants may be present in an amount ranging from 0.1 to 5 wt. %. This range includes all values and subranges therebetween, including 0.1, 0.2, 0.3, 0.4, 0.5, 0.6, 0.7, 0,8, 0.9, 1, 2, 3, 4, and 5 wt. %.

The composition may include one or more disintegrants. Some non-limiting examples of disintegrants include corn starch, potato starch, croscarmellose, crospovidone, sodium starch glycolate, polyvinylpyrollidone, Veegum HV, methylcellulose, agar, bentonite, cellulose, sponge, alginic acid, guar gum, citrus pulp, carboxymethylcellulose, and the like. Combinations are possible. The disintegrant or disintegrants may be present in an amount ranging from 1 to 15 wt. %. This range includes all values and subranges therebetween, including 1, 2, 3, 4, 5, 6, 7, 8, 9, 10, 11, 12, 13, 14, and 15 wt. %.

The composition may contain one or more coloring agents, flavoring agents, and the like. Combinations are possible.

The term “unit dose” or “unit dosage form” as used herein refers to physically discrete units of such composition suitable for use as unitary dosages by a human subject. Each unit contains a predetermined quantity of active agent or agents calculated to produce the desired treating effect.

The term “pharmaceutically acceptable,” as in a pharmaceutically acceptable carrier or excipient, refers to a carrier or excipient that has met the required standards of toxicological and manufacturing testing or that it is included on the Inactive Ingredient Guide prepared by the U.S. Food and Drug administration. In one embodiment, the excipients, e.g., diluent, binder, glidant, lubricant, and/or disintegrant are pharmaceutically acceptable.

“Pharmacologically active” (or simply “active”) as in a “pharmacologically active” derivative or analog, refers to a derivative or analog having the same type of pharmacological activity as the parent compound and preferably, but not necessarily, approximately equivalent in degree. In one embodiment, the curcumin compound, quercetin, papain, and bromelain are active agents.

The composition may be administered orally once or more than once as appropriate. If administered more than once, the composition may be administered on a regular basis or on an irregular basis. The composition may be administered at a rate of one to four times over a time period ranging from a single day to thirty days, optionally repeating as necessary, and optionally with one or more intervals of non-administration. These ranges include all values and subranges therebetween, including, for example, 1, 2, 3, and 4 times for administration, and a time period of 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30 days.

The total daily dose of active agent or agents may suitably range from about 500 mg to about 4000 mg, which range includes all values and subranges therebetween, including, for example, about 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3500, and 4000 mg, and any combination thereof. The doses herein are suitable whether for therapeutic or prophylactic administration and, in any case, may be suitably adjusted depending on the desired treating effect.

The values and subranges cited herein are set out for illustration purposes only, and are not intended to limit the available points within the range.

The present invention may be embodied in many different forms, and several embodiments are described herein in detail. It should be understood, however, that the present disclosure and the embodiments described herein are to be considered as exemplifications of the invention and are not intended to otherwise limit the invention, which is defined by the claims herein.

What is claimed is:

1. A composition, comprising:
50-80 wt. % of at least one curcumin compound;
10-50 wt. % quercetin;
≧3 wt. % papain; and
≧3 wt. % bromelain;
wherein the composition is suitable for oral administration to a human.

2. The composition of claim 1, wherein the curcumin compound is selected from the group consisting of bisdemethoxycurcumin, demethoxycurcumin, curcumin, dihydrocurcumin, tetrahydrocurcumin, hexahydrocurcumin, octahydrocurcumin, diacetylcurcumin, p, p dihydroxydicinnamoylmethane, hexahydrocurcuminol, curcumin glucuronide, curcumin sulfate, [6]-paradol, dihydrocapsaicin, salicylcurcuminoid, monomethylcurcumin, trimethylcurcumin, 1,7-bis(3,4-dihydroxyphenyl)-11,6-heptadiene-3,5-dione, p-dyhydroxydicinamoyl feruloyl methane, capsaicin, [6]-gingerol, cassumunin A, cassumunin B, yakuchinone A, yakuchinone B, isoeugenol, dibenzoylmethane, rosmarinic acid, 2-hydroxydibenzoylmethane, hydrazinocurcumin, hydrazinodemethoxycurcumin, hydrazinobisdemethoxycurcumin, hydrazinobenzoyldemethoxycurcmin, and a combination thereof.

3. The composition of claim 1, wherein the curcumin compound is selected from the group consisting of bisdemethoxycurcumin, demethoxycurcumin, curcumin, and a combination thereof.

4. The composition of claim 1, which comprises about 60-70 wt. % of the curcumin compound.

5. The composition of claim 1, which comprises about 65 wt. % of the curcumin compound.

6. The composition of claim 1, wherein the curcumin compound is a combination of bisdemethoxycurcumin, demethoxycurcumin, and curcumin.

7. The composition of claim 1, which comprises about 15-45 wt. % quercetin.

8. The composition of claim 1, which comprises about 25-40 wt. % quercetin.

9. The composition of claim 1, which comprises about 25-30 wt. % quercetin.

10. The composition of claim 1, which comprises about 3.1-20 wt. % papain.

11. The composition of claim 1, which comprises about 3.1-5.1 wt. % papain.

12. The composition of claim 1, which comprises about 3.1-10 wt. % bromelain.

13. The composition of claim 1, which comprises about 3.1-5.1 wt. % bromelain.

14. The composition of claim 1, further comprising one or more selected from the group consisting of cranberry, green tea extract, black cohosh, wood betony, passion flower, valerian root, saw palmetto, zinc, calcium, and a combination thereof.

15. The composition of claim 1, further comprising a sustained release coating.

16. A method, comprising contacting:
50-80 wt. % of at least one curcumin compound;
10-50 wt. % quercetin;
≧3 wt. % papain; and
≧3 wt. % bromelain;
to form a composition suitable for oral administration to a human.

17. The method of claim 16, wherein the composition is in the form of a capsule.

18. A method for treating one or more selected from the group consisting of chronic nonbacterial prostatitis, chronic pelvic pain syndrome, overactive bladder, sexual dysfunction, prostatitis, prostatodynia, urinary incontinence, pain during intercourse, genitourinary pain not associated with infection, bladder pain, abdominable pain, urinary frequency, category III chronic prostatitis syndrome, category IIIa chronic pelvic pain syndrome, category IIIb chronic pelvic pain syndrome, chronic voiding symptoms not associated with infection, adverse side effects associated with renal transplantation, protection against kidney damage associated with the administration of aminoglycosides, delayed graft function in kidney transplant patient, acute rejection in kidney transplant patient, drug induced neuropathy in kidney transplant patient, drug induced tremor in kidney transplant patient, chronic allograft nephropathy in kidney transplant patient, post-transplant risk of colon cancer in kidney transplant patient, post-transplant risk of prostate cancer in kidney transplant patient, protection against kidney damage from intravenous contrast dye, protection against ischemic kidney damage from trauma or surgery, hypercholesterolaemia, high LDL cholesterol level, and a combination thereof, comprising administering the composition of claim 1 to a human.
